(19) United States
(12) Patent Application Publication
(10) Pub. No.: US 2016/0106531 A1
(43) Pub. Date: Apr. 21, 2016

(54) INTRAVASCULAR DEVICES

(71) Applicant: KEYSTONE HEART LTD., Caesarea (IL)

(72) Inventor: Yuval SHEZIFI, Haifa (IL)

(73) Assignee: Keystone Heart Ltd., Caesarea (IL)

(21) Appl. No.: 14/892,802

(22) PCT Filed: May 18, 2014
(86) PCT No.: PCT/IL2014/050432

§ 371 Date: Nov. 20, 2015

Related U.S. Application Data
(60) Provisional application No. 61/826,012, filed on May 21, 2013.

(51) Int. Cl. A61F 2/01

(52) U.S. Cl. CPC A61F 2/01; A61F 2250/0098; A61F 2002/011; A61F 2220/0033; A61F 2230/0008

(57) ABSTRACT

The invention features intravascular devices, in particular devices for deflecting emboli (100), and elements for connecting such devices to wires. Such connecting elements are configured to limit within a range the relative rotation between device and wire.

### CROSS-REFERENCE TO RELATED APPLICATION

This application claims benefit of U.S. Provisional Application No. 61/826,012 filed May 21, 2013, which is hereby incorporated by reference in its entirety.

### FIELD OF THE INVENTION

Embodiments of the invention relate to deflecting emboli in an aorta to prevent emboli from entering arteries, for example, arteries that lead to the brain.

### BACKGROUND OF THE INVENTION

Devices such as vascular filters or other devices may be inserted into a blood vessel prior to or during a procedure or at another time. Such devices may be inserted by way of a catheter that may be, for example, threaded through a vein or artery, and into, for example, an aorta or other vessel where the device may be released from the catheter and, for example, deployed. The device may filter, deflect, or block emboli or other objects from entering into a blood supply that feeds the brain.

### SUMMARY OF THE INVENTION

In one aspect, the invention features an intravascular device including a first wire, a second wire, and a connecting element; wherein:

a. the connecting element includes a hollow cylindrical body defining an internal channel along a longitudinal axis;

b. the connecting element joins the first wire and the second wire;

c. the connecting element includes a first stop element;

d. the second wire includes a second stop element; and

e. the first stop element is configured to reversibly engage the second stop element such that the second wire is only able to freely rotate relative to the connecting element over a distance of between 10 and 360 degrees. For example, the device can freely rotate relative to the wire approximately 10, 20, 30, 40, 50, 60, 70, 80, 90, 120, 145, 160, 180, 210, 240, 270, 300, 330, or slightly less than 360 degrees (e.g., the wire can rotate at least 10, 20, 30, 40, 50, 60, 70, 80, 90, 120, 145, 160, 180, 210, 240, 270, 300, 330, or slightly less than 360 degrees or at most 10, 20, 30, 40, 50, 60, 70, 80, 90, 120, 145, 160, 180, 210, 240, 270, 300, 330, or slightly less than 360 degrees).

In some embodiments, the hollow cylindrical body includes a window having two edges substantially parallel to the longitudinal axis and the edges reversibly engage the second stop element thereby defining the first stop element. In related embodiments, the first stop element is disposed within the window. In other embodiments, the first stop element is disposed on the interior of the hollow cylindrical body.

In the above and related embodiments, the second stop is a protrusion disposed on the surface of the second wire. Alternatively, the second stop element includes a substantially cylindrical element that is disposed about the second wire or joined to an end of the second wire, the substantially cylindrical element including a window having two edges substantially parallel to the longitudinal axis that reversibly engages the first stop element. In one particular embodiment, the second stop element includes a substantially cylindrical element that is disposed about the second wire or joined to an end of the second wire, the substantially cylindrical element including a window including:

(i) two edges substantially parallel to the longitudinal axis that reversibly engage the first stop element; and

(ii) a third edge substantially orthogonal the longitudinal axis.

This embodiment may also feature the joining of the second wire and the connecting element by bending the first stop such that it is disposed within the window in the substantially cylindrical element, thereby permitting reversible engagement with the second stop element. Here, for example, upon disposition within the window in the substantially cylindrical element, the second stop element reversibly engages the third edge, thereby preventing separation of the second wire and the connecting element.

In another aspect, the invention features a device for deflecting emboli, the device including:
a lateral structure to support an emboli filter, a length of the lateral structure being between 80 mm and 90 mm and a width of the lateral structure being from 20 mm to 35 mm, having the filter attached to and extending the length of the lateral structure;
a lower member extending downward from the lateral structure in a direction of an ascending aorta, wherein upon installation of the device, the lower member exerts lift on a middle area of the lateral structure; and
an upper member extending upwards from the lateral structure, wherein a support portion of the upper member being proximate to the lateral structure is angled towards the ascending aorta, and an anchor portion of the upper member being distal to the lateral structure is angled towards the descending aorta, wherein upon the installation, the upper member limits the lift;
wherein the device includes three or more radiopaque elements (e.g., a clamp, bead, or element incorporated into the wire of the intravascular device); and
wherein the three or more radiopaque elements are positioned asymmetrically arranged about the device, e.g., asymmetrically in 2 or more axes. Here, one or more radiopaque elements may optionally be incorporated into all or a portion of the wire forming the skeleton or filter mesh of the device.

In certain embodiments, the radiopaque elements are located exclusively at the following positions: a junction between the lateral structure and lower member, a junction between the lateral structure and upper member, at the top of the upper member, and, optionally, at the distal and proximate ends of the lateral structure.

### BRIEF DESCRIPTION OF THE DRAWINGS

FIG. 1A is a schematic diagram of a side view of an intra-vascular device in accordance with an embodiment of the invention.

FIG. 1B is a schematic diagram of a three-quarters view of an intra-vascular device, in accordance with an embodiment of the invention.

FIG. 2 is a schematic diagram of a device installed in an aorta, in accordance with an embodiment of the invention.

FIG. 3 is a diagram of a hooked end of a device with a contact point between a loop of wire and a rest of the hook, in accordance with an embodiment of the invention.

FIG. 4 is a flow diagram of a method in accordance with an embodiment of the invention.

FIG. 5A is a diagram of a three-quarters view of an intra-vascular device with a radiopacity bead, in accordance with an embodiment of the invention.

FIG. 5B is a photograph of a radiopacity bead and clamp element for use in an embodiment of the invention.

FIG. 5C is a photograph of a cross section of Drawn Filled Tubing (DFT wire).

FIG. 5D is a schematic diagram of a side view of an intra-vascular device, in accordance with an embodiment of the invention.

FIG. 5E is a schematic diagram of a filter mesh containing DFT wire.

FIG. 5F is a series of schematic diagrams showing a particular embodiment of the present invention in which five radiopaque elements are affixed to or incorporated into the skeleton of an intra-vascular device. All diagrams of FIG. 5F are directed to a single embodiment. Circles indicate the approximate location, but not necessarily the size, of radiopaque elements. In the diagrammed embodiment, the radiopaque elements are positioned at each of the length-wise tips of the intra-vascular device, at the top of the upper member, at a position on the left upper member proximal to the junction of the left upper member, and at a position on the left lower member proximal to the junction of the left lower member. The position of a given radiopaque element in any particular diagram represents the location of that radiopaque element only in respect to the portrayed dimensions.

FIG. 5G is a series of schematic diagrams showing a particular embodiment of the present invention in which five radiopaque elements are affixed to or incorporated into the skeleton of an intra-vascular device. All diagrams of FIG. 5G are directed to a single embodiment. Circles indicate the approximate location, but not necessarily the size, of radiopaque elements. In the diagrammed embodiment, the radiopaque elements are positioned at each of the length-wise tips of the intra-vascular device, at the top of the upper member, at a position on the left upper member proximal to the junction of the left upper member, and at a position on the left lower member proximal to the junction of the left lower member. The position of a given radiopaque element in any particular diagram represents the location of that radiopaque element only in respect to the portrayed dimensions.

FIG. 6A is a photograph and a series of schematic diagrams showing various views of an intra-vascular device, in accordance with an embodiment of the invention.

FIG. 6B is a photograph and a series of schematic diagrams showing various views of an intra-vascular device, in accordance with an embodiment of the invention.

FIG. 6C is a schematic diagram showing a front view of an intra-vascular device, in accordance with an embodiment of the invention.

FIGS. 7A and 7B are schematic diagrams showing side views of intra-vascular devices, in accordance with embodiments of the invention. FIG. 7A illustrates a skeleton with an increased thickness or multiple wires in order to increase the skeleton stiffness when compared with the device of FIG. 7B.

FIG. 8A is a schematic diagram showing filter meshes of the indicated pore sizes.

FIG. 8B is a schematic diagram showing perforated films with the indicated patterns, sizes, and densities of pores.

FIG. 8C is a schematic diagram showing a filter mesh with a combination of DFT and Nitinol wires.

FIGS. 9A-9C are photographs showing a variety of mechanisms for connecting the intra-vascular device to a catheter.

FIG. 9D is a series of photographs depicting a connecting element in one embodiment of the invention. Here, the tether (substantially cylindrical element) and letch (second stop) are indicated.

FIGS. 9E and 9F are diagrams showing the embodiment depicted in FIG. 9D. The connector (A), tether (substantially cylindrical element; B) and letch (second stop; C) are labeled.

FIG. 9G is a photograph of the tether of the embodiment of FIG. 9D absent the rest of the connecting element.

FIGS. 9H-9L are a series of schematics showing various configurations of the embodiment of FIG. 9D where the connector and letch are rotated relative to the tether.

FIG. 10 is a schematic diagram of a side view of a plunger for use in introducing intra-vascular devices of the invention into a subject, e.g., through a catheter.

### DETAILED DESCRIPTION OF THE PREFERRED EMBODIMENTS

In the following description, various embodiments of the invention will be described. For purposes of explanation, specific examples are set forth in order to provide a thorough understanding of at least one embodiment of the invention. However, it will also be apparent to one skilled in the art that other embodiments of the invention are not limited to the examples described herein. Furthermore, well-known features or processes may be omitted or simplified in order not to obscure embodiments of the invention described herein.

In general the invention features an element for connecting an intravascular device (e.g., the devices described in International Application Number PCT/IL2011/000963, incorporated by reference in its entirety) to a wire. The invention also features certain arrangements of radiopaque elements on intravascular devices, e.g., the devices described in International Application Number PCT/IL2011/000963.

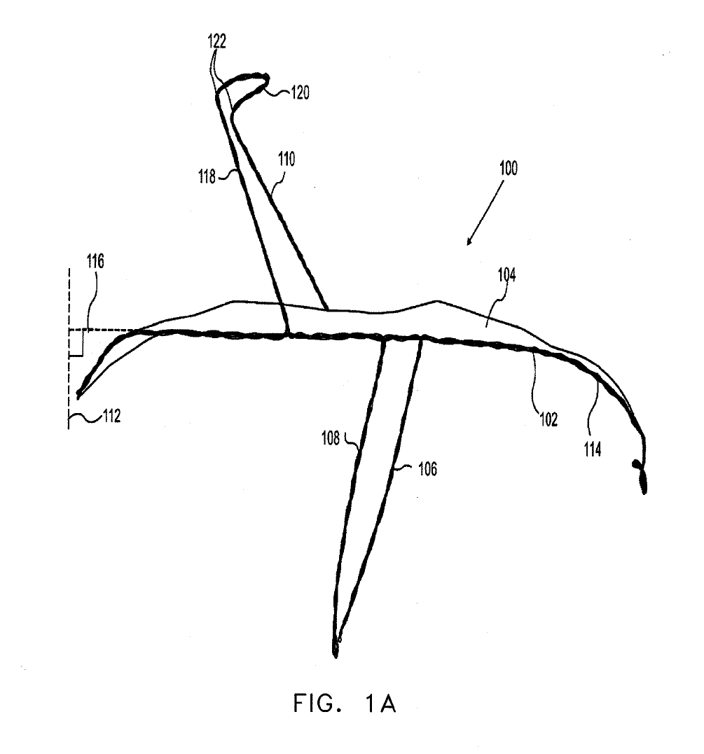

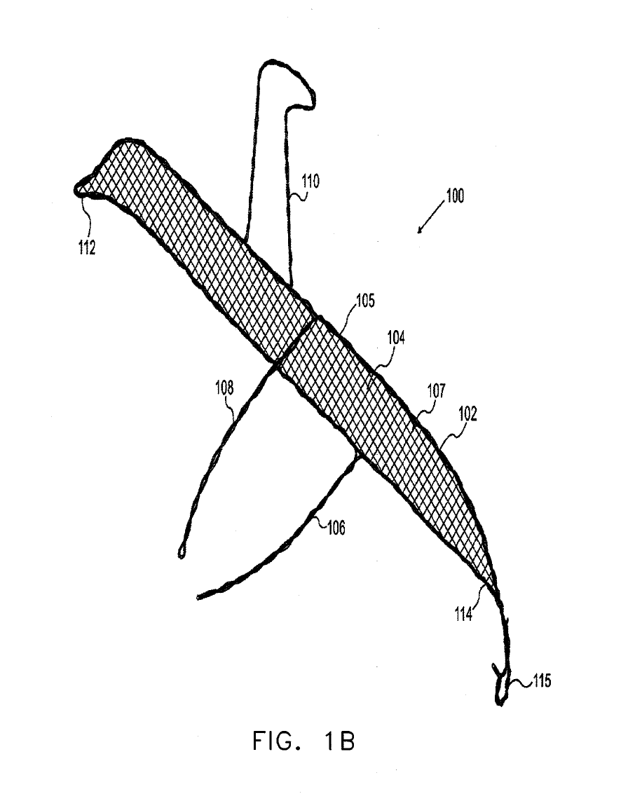

Reference is made to FIG. 1A, a schematic diagram of a side-view of an intra-vascular device, and to FIG. 1B, a three quarters side view of an intra-vascular device, in accordance with an embodiment of the invention. In some embodiments, an intravascular device 100 may include a lateral structure such as a frame or skeleton 102, a filter 104, and a series of support members such as lower members 106 and 108, and upper member 110. A first end 112 of device 100, facing upstream of blood flow in an aorta, and a second end 114 of device 100, facing downstream of blood flow in an aorta, may curve downward below a lateral plane of device 100. Second end 114 of device 100 may include a hook 115 or other clasping and retrieval section by which device 100 may be held on insertion or installation, and then snared or grasped for retrieval of device 100. In certain embodiments, the second end 114 of device 100 may include a clasp that remains connected throughout introduction and removal from a subject's aorta.

Imaginary line 116 represents a theoretical lateral plane of device 100. In some embodiments, a lateral plane of device 100 may include an approximately horizontal line tracing a middle section of skeleton 102 along device 100 before the curves of end 112 and end 114.

A first support portion 118 of upper member 110, as may be proximate to skeleton 102, may rise away from skeleton 102 at an angle towards first end 112. A second anchor portion 120 of upper member 110 may double back on such first support portion at bend 122 and may rise upward and towards a direction of second end 114. Second anchor portion 120 of upper member 110 may taper in width towards its tip, which may be rounded or flattened.

In some embodiments, a weave of strands 107 of filter 104 may be angled at approximately 45 degrees to an outside of skeleton 102 to accommodate shifts in length or with of the structure of skeleton 102 when device 100 is installed, removed or positioned into place.

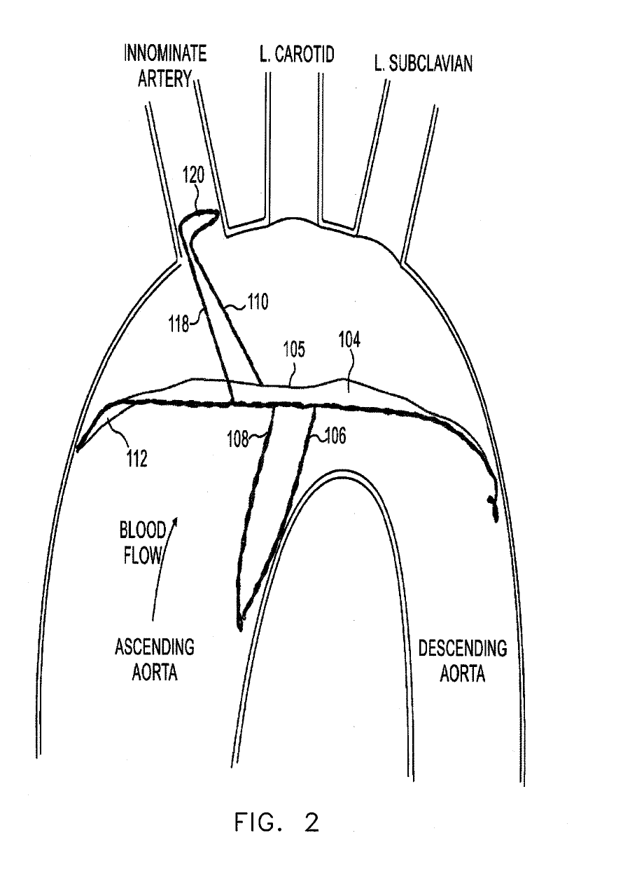

Reference is made to FIG. 2, a schematic diagram of a device installed in an aorta, in accordance with an embodiment of the invention. In operation, device 100 may be installed in an aorta such that first end 112 points towards the ascending aorta. Lower members 108 and 106 may press against an internal lower wall of the ascending aorta, and such pressing may exert an upward lift force on a middle portion 105 of skeleton 102. Such lift may raise middle portion 105, and, for example, a majority, or some other portion of skeleton 102, above lateral plane 116 of skeleton 102, or may stop such middle portion 105 from sinking below lateral plane 116 of device 100. In some embodiments, lift from lower members 106 and 108 may be exerted only at such time as some other force, such as a blood flow or pulse movement, acts to push device 100 downward below lateral plane 116.

Upon deployment, installation or release, upper member 110 may extend into an innominate artery. For example, first support portion 118 of upper member 110 may come into contact with a right internal wall of the innominate artery, bend 122 may come into contact with a right internal wall of the innominate artery, and second anchor portion 120 of upper member 110 may come into contact with a superior portion of a left internal wall of the innominate artery. The multiple, possible contact or holding points of upper member 110 with the innominate artery may hold device 100 in place against a blood flow in the aorta, may prevent a roll of device 100 within the aorta, and may prevent device 100 from rising beyond a desired distance from an entry point of the innominate, left carotid and left subclavian arteries. Upper member 110 may also prevent device 100 from sliding out of position in a direction of a blood flow or of reverse flow in the aorta. Upper member 110 may exert a downward force on device 100 to counter a lift that may be exerted by lower members 106 and 108, and to keep device 100 away from the entry points of the branch arteries of the aorta, for example, as are listed above.

In some embodiments, upper member 110 may be inserted into, for example, a left subclavian artery where a curve of bend 122 may be held against a left inner wall of left subclavian artery, and second anchor portion 120 engages a counter wall.

The downward curve of first end 112 and second end 114 may likewise press against an ascending aorta and descending aorta respectively, to prevent a rise of device 100 past a desired position that approximates a midway between a lower wall of the aortic arch and an upper wall of the aortic arch. The downward curve of first end 112 and second end 114 may allow pressure to be exerted against walls of the aorta without damaging or puncturing such walls. Lower members 106 and 108 may exert a continuous lift force on skeleton 102 to keep first end 112 and second end 114 in pressure contact with an upper wall of the ascending aorta and descending aorta respectively.

In an installed position, mesh or filter 104 may block or deflect emboli or other particles from entering, for example, the three branch arteries listed above, while still preserving a space above the filter for blood to swirl and collect at such entries. The space under filter 104 may allow unfiltered blood to pass by the branch arteries of the aorta. Such space in the aorta that is left below the filter means that not all blood passing through the aorta is subject to the filtering or deflecting process of filter 104. Installation in a middle (such as between an upper wall of the aortic arch and a lower wall of the aortic arch) of the aorta rather than directly abutting an entry point into the branch arteries may allow a continued flow of blood both through the aorta and into the branch arteries, even if a portion of filter 104 is clogged with embolic or other material.

In some embodiments, lower member 106 may be connected to skeleton 102 on a first side (such as a dorsal side), and lower member 108 may be connected to skeleton 102 on a second side (such as a ventral side). A first portion of each of lower member 106 and lower member 108 that are proximate to skeleton 102 may extend in substantially parallel lines from skeleton 102. A second or lower portion of each of lower members 106 and 108, as are distal to skeleton 102 may curve towards each other at a point approximating a mid-line of skeleton 102. The lower ends of lower members 106 and 108 may terminate in, for example, small loops of the single wound strand that each of the members includes. Such curved endings may prevent a scratching or abrasion of an end of the lower member 106 or 108 against arterial tissue. The ends of each of lower members 106 and 108 may in some embodiments touch gently together though they may separate with light pressure.

In some embodiments device 100 may remain positioned in an aorta while a procedure (e.g., transcatheter aortic valve implantation) is undertaken in, for example, a heart, blood vessel, or other in-vivo area, where such procedure entails tracing a lead such as a catheter through the aorta. The ease of separation of lower members 106 and 108 may allow a removal of an arterial catheter or other device from the aorta while device 100 remains in place, and serves to deflect or filter embolic material away from entering branch arteries of the aorta.

Reference is made to FIG. 3, a diagram of a hooked end of a device with a contact point between a loop of wire and a rest of the hook, in accordance with an embodiment of the invention. In some embodiments, hook 115 may include a latch 300 or wire strand that may be part of a wire strand that makes up skeleton 102, and that is in contact with a rest of hook 115. In some embodiments, a wire or catheter that may end in, for example, a loop, may be threaded through latch 300 so that the loop passes between a contact point of bend 302 and curve 304. When so threaded, a wire or catheter fitted with a looped end may be clicked into hook 115, and may securely push device 100 into place or pull device out of position from an aorta. In some embodiments, the hook may end in a ball-tip so that strands from the frame do not fray or scratch the vessel wall or the inner tube of a catheter.

In some embodiments, device 100 may prevent the passage of, block, divert, or filter-out particles, such as, for example, blood clots, calcified debris or other objects that may block a flow of blood. Skeleton 102 and device 100 may also be used to support or keep in place other apparatuses. In some embodiments, device 100 may be inserted into a vessel by way of, for example, a catheter, and may be threaded into, for example, a blood vessel into which device 100 may be implanted. Other methods of implanting device 100 into a blood vessel are possible. In some embodiments, device 100 may assume a shape of an extended oval or a willow leaf. Other shapes may be used.

In some embodiments, skeleton 102 may include or be constructed of, for example, Nitinol or other superelastic or shape memory alloy or material. Other materials may be used. In some embodiments, filter 104 may be or include a fine wire netting or mesh, or perforated film, such as a mesh having holes or pores of 300 microns more or less such that, for example, particles that are larger than the pores or holes are prevented from passing through the filter. Other sizes of holes or eyes may be used. In some embodiments, a shape of filter 104 may be defined or supported by a shape of skeleton 102.

In some embodiments, one or more of skeleton 102, upper member 110 and lower members 106 and 108 may be fashioned of continuous wire that has different thicknesses or properties in various areas of its lengths. For example, upper member 110 may be fashioned of a wire or portion of wire that is thin or otherwise highly flexible relative to the thickness or flexibility of one or more of lower members 106 and 108 or of other portions of skeleton 102. Such heightened flexibility may enable upper member 110 and particularly bend 122 and second portion 120 to expand or shrink upon the application of even a small force, such as, for example, the small force exerted by the contact of upper member 110 with an upper portion of a blood vessel against which it comes into contact. In contrast, lower members 106 and 108 may be fashioned of a thicker or relatively more rigid wire or filament to provide lift for a mid portion of device 100.

In some embodiments, one or more of the wires that make up upper member 110 and lower members 106 and 108 may be wound or braided around skeleton 102, and no soldered or glued connections between the wound strands of skeleton 102 and members 110, 106 and 108 may be needed.

In some embodiments, device 100 may be inserted or deployed through, for example, one of the branch arteries or directly through an artery in the area of the heart rather than by way of a catheter from a remote vessel.

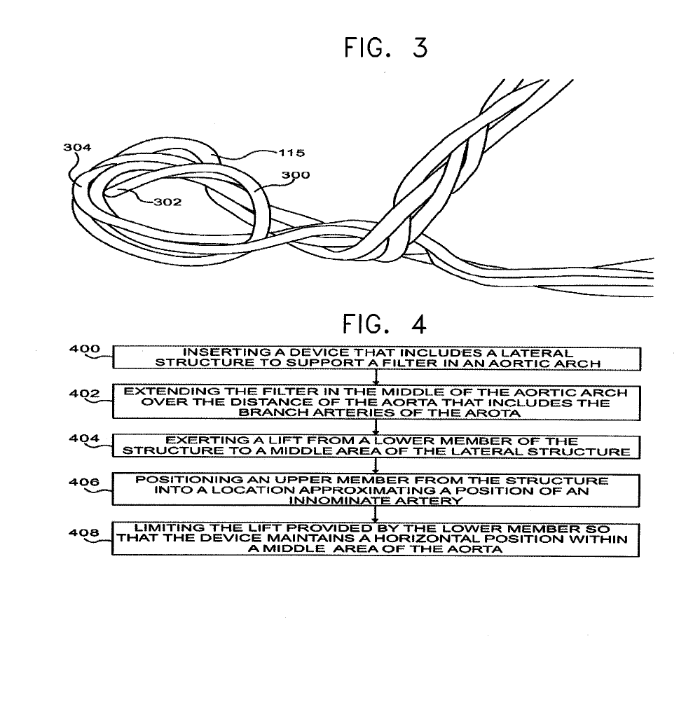

Reference is made to FIG. 4, a flow diagram of a method in accordance with an embodiment of the invention. In block 400, there may be inserted into an aortic arch, a device that includes a lateral structure to support a filter (for example, the device of FIGS. 1-3, 5A, 6A-6C, 7A, or 7B may be used, or other devices described herein may be used). The length of the device may be from approximately 80 mm to 90 mm, or otherwise as may be necessary to approximate a distance between an upper wall of an ascending aorta, upstream of an opening of an innominate artery, and at an upper wall of a descending aorta downstream of an opening of a left subclavian artery. The width of the device may be from 20 mm to 35 mm, or otherwise as may approximate an internal diameter of an aorta. The device may be inserted into the aorta or introduced into a blood vessel in a collapsed form, and may assume an extended form upon its release from a tube or other insertion or positioning mechanism. In block 402, the device may extend the filter attached to the lateral structure so that the filter assumes a position approximately midway between an upper wall of the aortic arch and a lower wall of the aortic arch, and extends over the distance between the branch arteries of the aorta as are listed in block 400 above.

In block 404, a lower member connected with the device may extend downward from the lateral structure in a direction of an upstream blood-flow, and such lower member may exert a lift on a middle area of the lateral structure.

In block 406, an upper member that may be connected to the structure, may be angled in a proximate section towards upstream flow of blood in the aorta, and in a distal section relative to the device, may be angled towards downstream flow of blood in the aorta, where a bend in such upper member between the support portion and the anchor portion, may extend into a location approximating a position of an innominate artery.

In block 408, the upper member may limit the lift provided by the lower member so that the device maintains a relatively horizontal position within a middle area of the aorta.

In some embodiments, the structure is bent downward from its lateral plane on each of a first end and second end of the structure, and outward force is exerted from each of such first end and a second end of the structure upon an inner wall of an ascending aorta and descending aorta, respectively.

In some embodiments, the embolic material is filtered from entering the branch arteries of the aorta.

In some embodiments, a method may include snaring a hook at a downstream end of the device with a loop brought into contact with the hook.

In some embodiments, a method may include separating a contact of a first side of the lower member from a second side of the lower member by pulling another device through the aorta to an area bounded by the lateral structure and the first and second sides of the lower member.

In some embodiments, a method may include bending an inferior or lower portion of the first side of the lower member towards an inferior or lower portion of the second side of the lower member.

In some embodiments, the invention may feature a method of manufacturing a device of the invention by tapering or narrowing the distal end of the upper member after the bend of the upper member so that the upper member ends in a narrow, curved point.

In some embodiments, a method may include winding a strand of structural material around the lateral structure, and extending such wound strand into one or more of the upper and lower members respectively. In some embodiments, the entire frame may be fabricated from a foil sheet that includes both the frame and the filter.

In particular embodiments, the present intra-vascular device may include elements that permit the determination of the orientation of the intra-vascular device in three-dimensional space. For instance, the intra-vascular device may include elements that allow the orientation of device to be determined when the device is present within a subject. The capacity to reveal the orientation of the intra-vascular device even when visual contact with the device is not available may be of value to practitioners.

In certain embodiments, the intra-vascular device may include radiopaque elements positioned such that, when the positions of some or all of the radiopaque elements are known, the orientation of the intra-vascular device in three-dimensional space may be determined In particular embodiments, the intra-vascular device may include three or more radiopaque elements. For instance, the intra-vascular device may include 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 or more radiopaque elements.

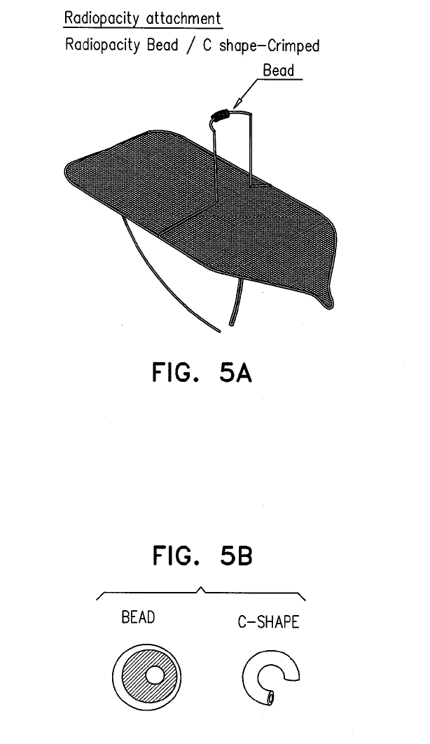

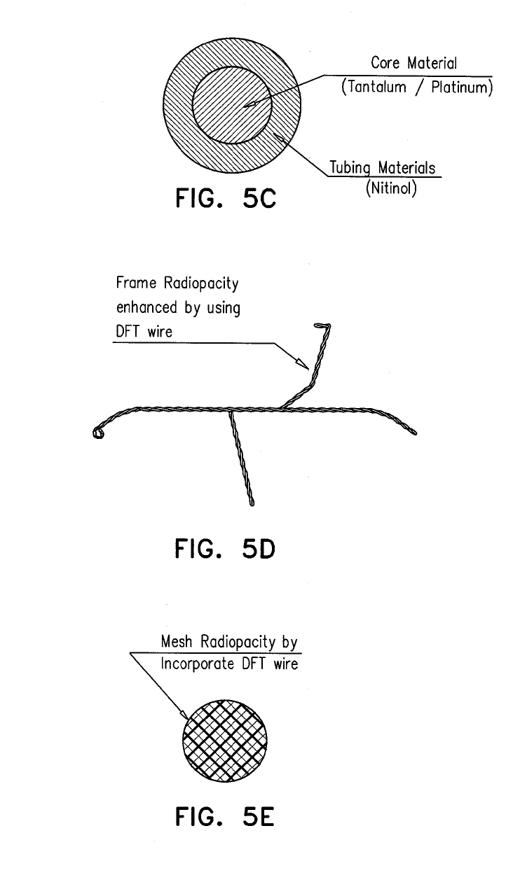

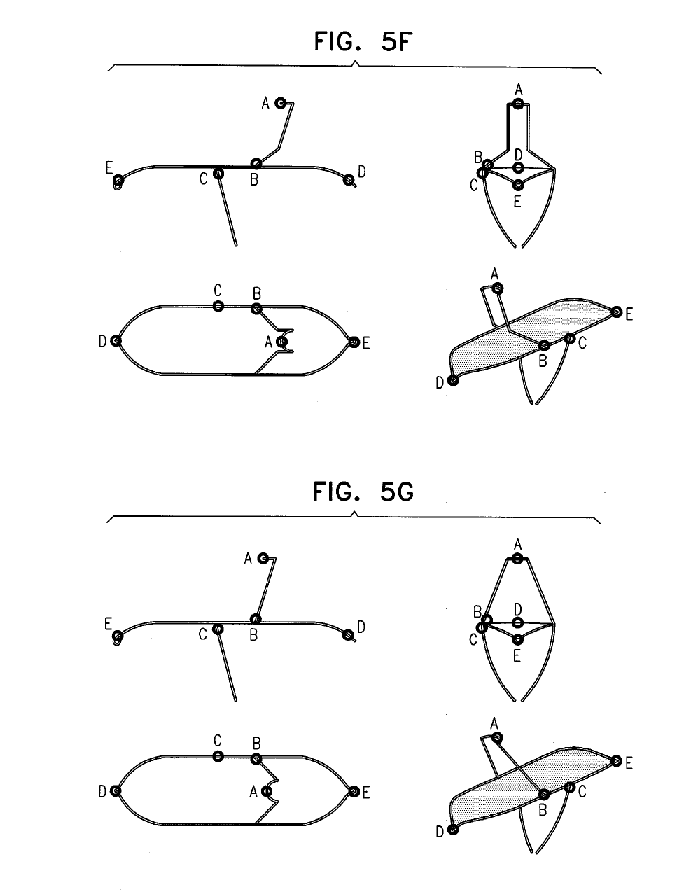

In some embodiments of the present invention, radiopaque elements are positioned such that a particular orientation of the radiopaque elements could equate to one and only one orientation of the intra-vascular device. When the present intra-vascular device includes three or more radiopaque elements, the elements may be spatially arranged in an asymmetric manner with respect to at least one axis or dimension. For instance, the radiopaque elements may be spatially arranged in an asymmetric manner with respect to one, two or three axes or dimensions. In particular embodiments, the radiopaque elements are asymmetric with respect to two or three axes or dimensions (e.g., as depicted in FIG. 5E or 5F). Referring to FIGS. 5E and 5F, radiopaque elements can be located at approximately (A) and either (B) or (C); (A), (B), and (C); (A), (D) and either (B) or (C); (A), (E) and either (B) or (C); (A), (B), (C), and (D); (A), (B), (C), or (E); or (A), (B), (C), (D) and (E). In another embodiment, elements (B) and/or (C) can be located on the opposite side of the filter frame.

A radiopaque element of the present invention may be a radiopaque clamp or bead affixed to or incorporated into the intra-vascular device. In the case of a clamp, the element can be crimped onto the intra-vascular device. A radiopaque element may be affixed to or incorporated into any aspect of the intra-vascular device. For instance, a radiopaque element such as a radiopaque bead or clamp may be an element affixed to or incorporated into the skeleton of the intra-vascular device. In particular embodiments, one or more beads or clamps may be affixed to or incorporated into one or more of the top of the upper member, either or both of the left and right aspects of the upper member, either or both of the left and right lower members, a tip of the intra-vascular device, the filter skeleton, clasp, or the filter material, e.g., mesh material. A radiopaque element may be proximal to or distal from a junction of the intra-vascular device, or at an extremity of the intra-vascular device. It will be understood by those of skill in the art that the precise locations or distribution of the radiopaque elements will not determine the utility of the radiopaque elements.

A radiopaque element may be an element affixed to or incorporated into the wire forming the intra-vascular device or the filter mesh of the intra-vascular device. A radiopaque element may be a radiopaque wire such as a Drawn Filled Tubing (DFT wire). Such wire can contain, e.g., a core of tantalum and/or platinum and an outer material of, e.g., Nitinol (see, e.g., FIG. 5C). In certain embodiments, the DFT wire can be incorporated into all or a portion of the intra-vascular device skeleton, upper member (e.g., as depicted in FIG. 5D), either or both of the lower members, clasp, or filter mesh. In embodiments where radiopaque wire (e.g., DFT wire) is used in the filter mesh, it can be used throughout the mesh, or in a certain subset of mesh wires (e.g., as depicted in FIG. 5E).

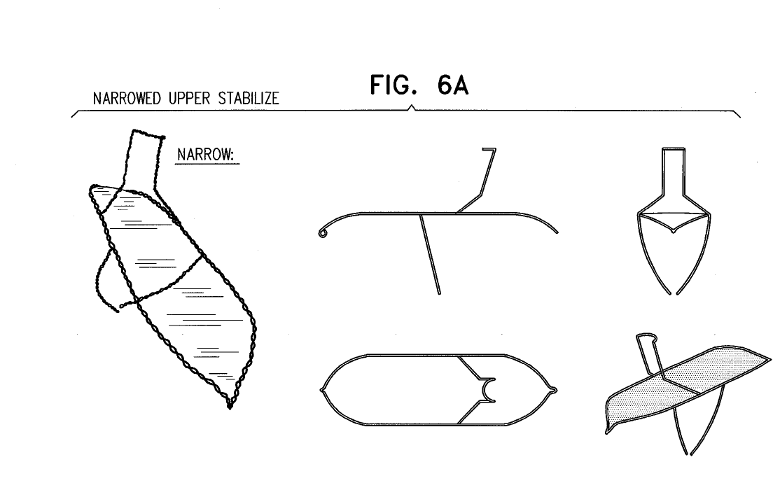

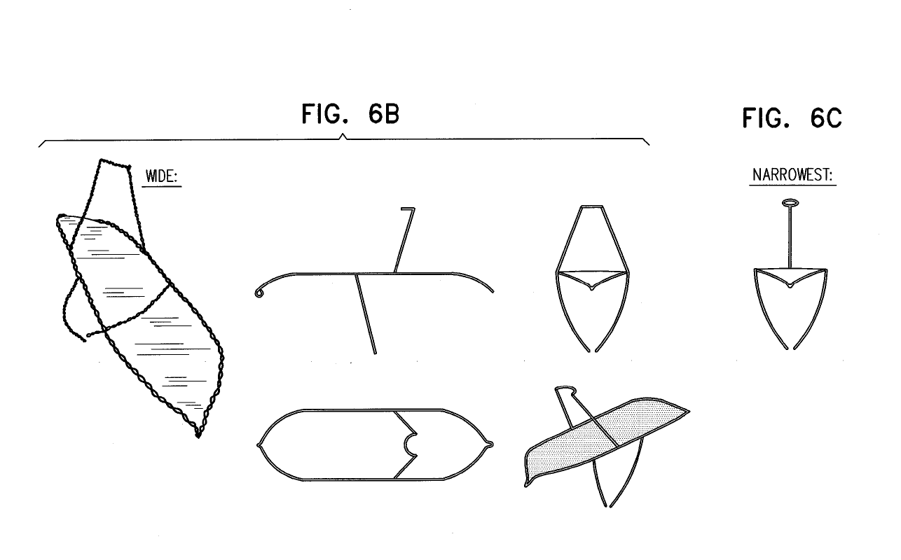

Reference is made to FIGS. 6A-6C. A variety of upper member structures can be used in the intra-vascular devices of the invention. Because the aortic anatomy can vary between individuals, embodiments of the intra-vascular device of the invention are shaped to adapt to a variety of aortic anatomies. FIGS. 6A-6C depict three different configurations for an upper member to be used in the intra-vascular devices of the invention. Each configuration contains a support portion and an anchor portion. Generally, the anchor portion will have a smaller width than the widest region of the lateral structure of the intra-vascular device. The anchor portion can have a width, e.g., of 100%, 95%, 90%, 85%, 80%, 75%, 70%, 60%, 50%, 40%, 30%, 20%, 10%, or less of the width of the widest region of the lateral structure of the intra-vascular device (e.g., the width can be between 100% and 10%, 80% and 20%, 60% and 20%, 50% and 20%, and 50% and 30% of the widest region of the lateral structure of the intra-vascular device). The anchor portion can be connected (e.g., directly connected) to a support portion of the upper member. In some embodiments, the anchor portion can be connected to a single support member of the support portion (e.g., as depicted in FIG. 6C) or can be connected to multiple support members (e.g., as depicted in FIGS. 6A and 6B). The anchor portion can, e.g., connect two support members (e.g., as depicted in FIGS. 6A and 6B) or can be configured as a loop connected to a single support member (e.g., as depicted in FIG. 6C). In some embodiments, the support members can be sloped towards the anchor portion. These support members can have a uniform (e.g., as depicted in FIG. 6B) or non-uniform (e.g., as depicted in FIG. 6A) slope. In embodiments with a non-uniform slope, the support members will contain an inflection point. At the inflection point, the distance between the support members can have, e.g., a width of 100%, 95%, 90%, 85%, 80%, 75%, 70%, 60%, 50%, 40%, 30%, 20%, 10%, or less of the width of the widest region of the lateral structure of the intra-vascular device (e.g., the width can be between 100% and 10%, 80% and 20%, 60% and 20%, 50% and 20%, and 50% and 30% of the widest region of the lateral structure of the intra-vascular device). Furthermore, the anchor portion can have a width of 100%, 95%, 90%, 85%, 80%, 75%, 70%, 60%, 50%, 40%, 30%, 20%, 10%, or less of the distance between the inflection points of the two support members (e.g., the width of the anchor portion can be between 100% and 50%, 100% and 70%, 100% and 80%, 90% and 70%, and 90% and 80% of the distance between inflection points of the support members). In certain embodiments, the width of the anchor portion and the distance between the inflection points of the two supporting members can be approximately the same. In such embodiments, the portion of the support members distal to the lateral support side of the inflection point will have substantially parallel slopes. In certain embodiments, the distal and proximal segments of each support member form a medial angle of more than 180° (e.g., as depicted in FIG. 6A).

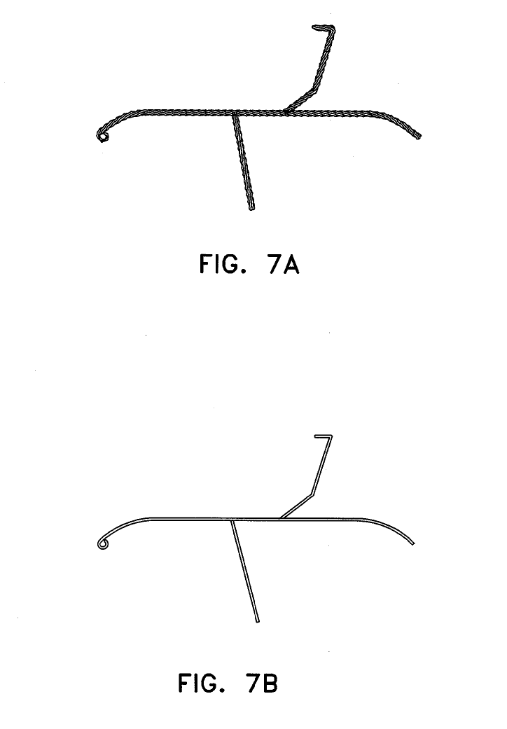

Reference is made to FIGS. 7A and 7B. In certain embodiments, the stiffness of the intra-vascular device will be determined by the thickness of the skeleton. For example, the skeleton can be stiffened by the inclusion of heavier gauge wire. Furthermore, multiple wires of a certain gauge can be wound together to increase the stiffness of the skeleton (e.g., the skeleton can include 2, 3, 4, 5, or more wires of to increase the stiffness of the intra-vascular device).

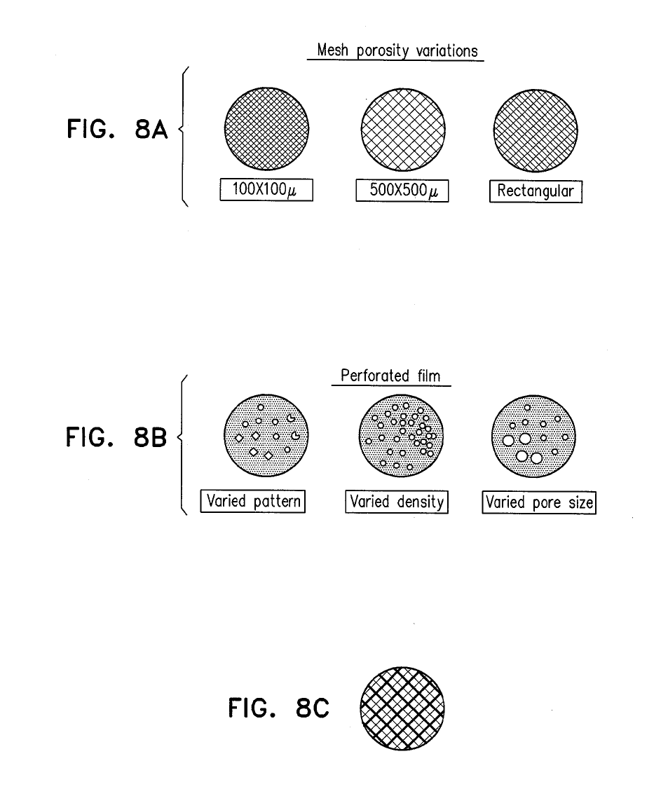

Reference is made to FIGS. 8A-8C. In certain embodiments of the intra-vascular device of the invention, the filter material can be a mesh (e.g., as depicted in FIGS. 8A and 8B) or a perforated film (e.g., as depicted in FIG. 8B). In embodiments where a wire mesh is used, the wire mesh can contain square, rectangular, or rhomboid shaped pores. Each dimension of the mesh pores can be, e.g., between 50 and 1000 microns (e.g., 100, 200, 300, 400, 500, 600, or more microns; FIG. 8A). In embodiments where a perforated film is present, the pores can have constant or varied pore patterns, constant or varied pore densities, and/or constant or varied pore sizes (FIG. 8B).

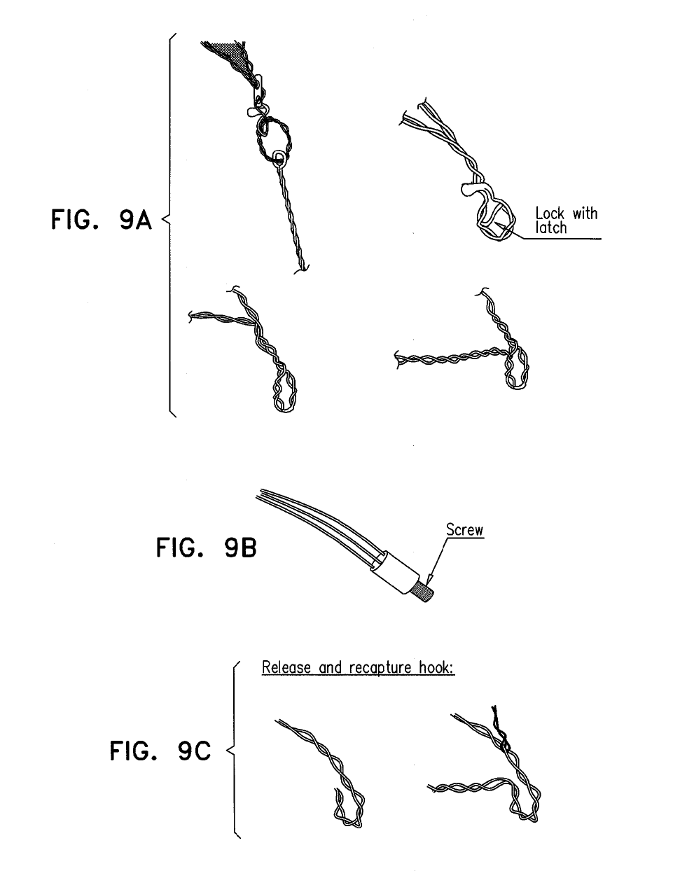

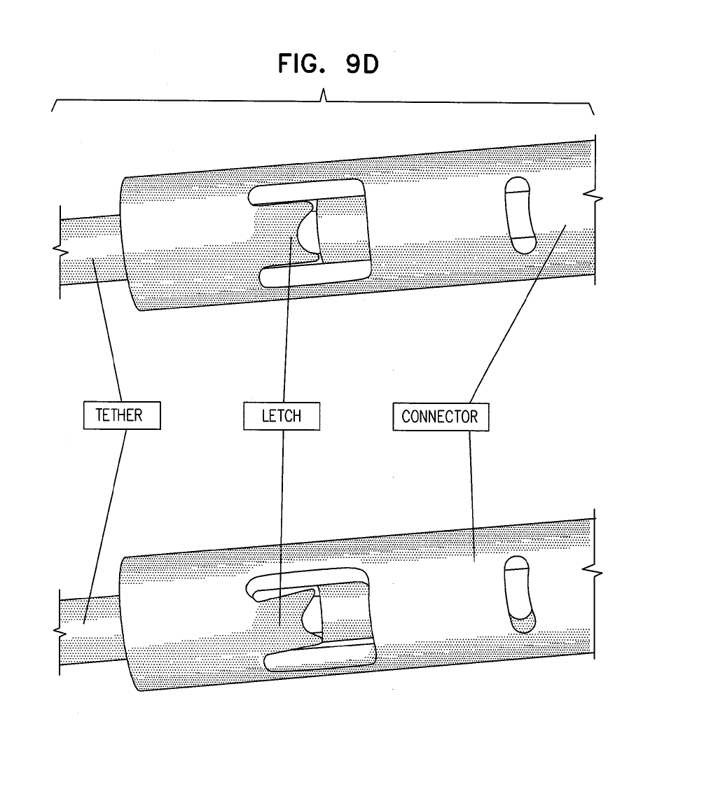

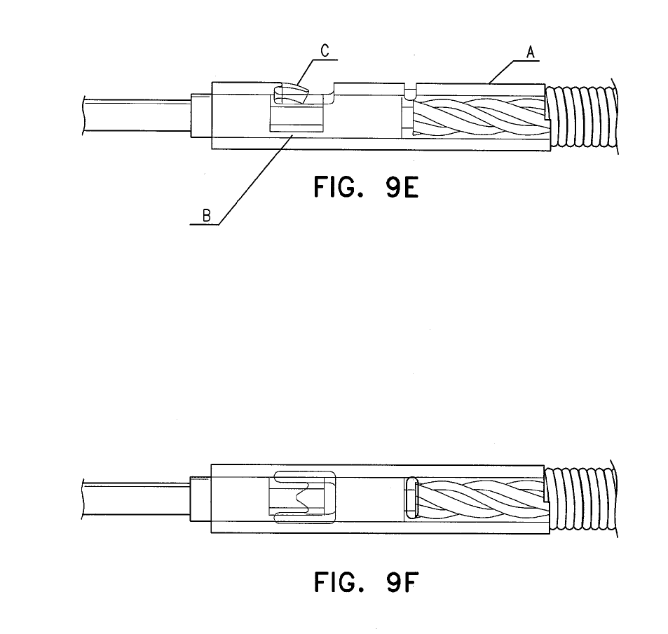

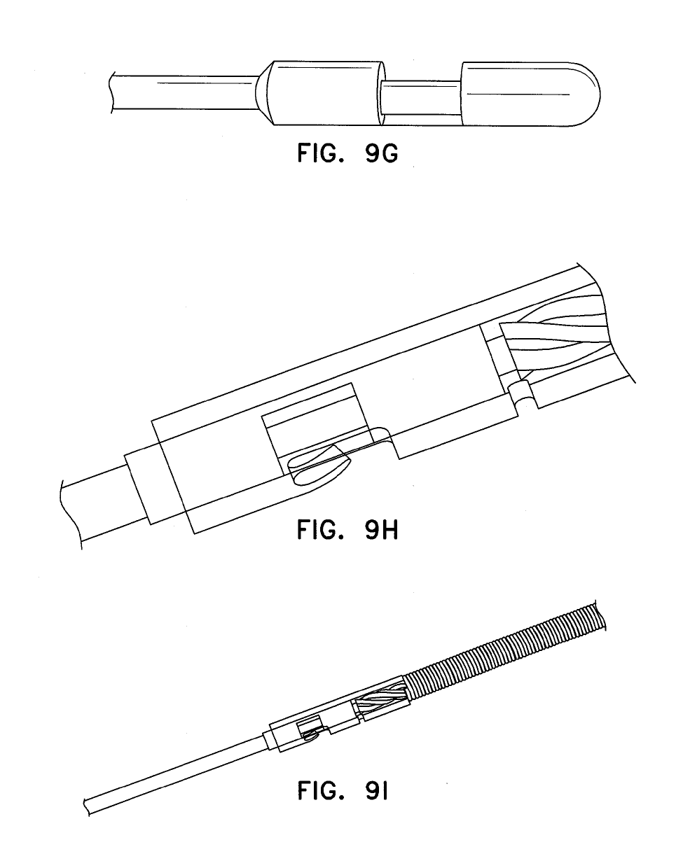

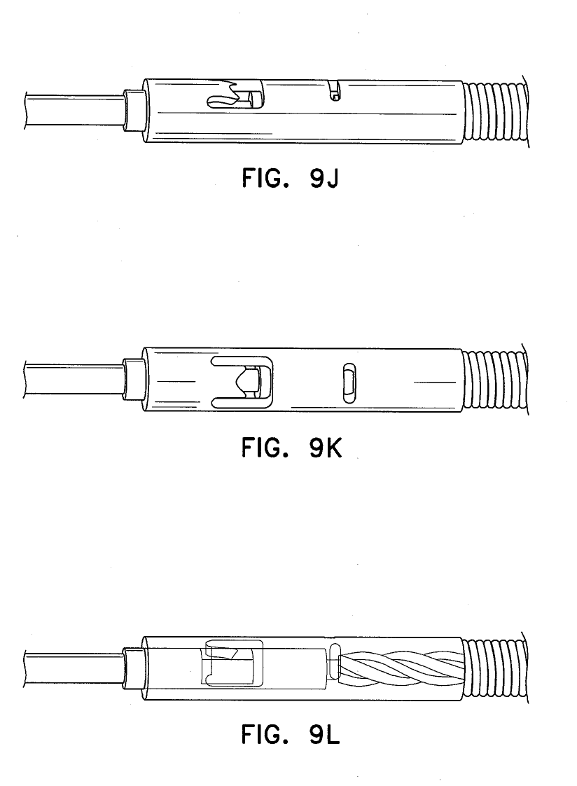

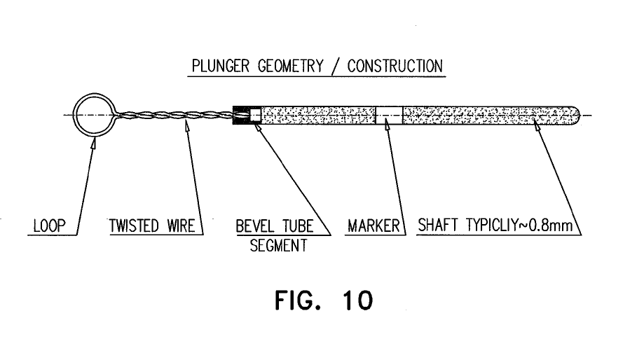

Reference is made to FIGS. 9A-9C. As described above, a variety of configurations can be used to connect the intra-vascular filter to a plunger (e.g., a plunger disposed within a catheter). FIG. 9A depicts a locking mechanism with a latch. FIG. 9B depicts a screw whereby the intra-vascular device can be mated with a screw on a plunger. FIG. 9C depicts a release and recapture hook for connecting the intra-vascular device with a plunger.

In one embodiment of the invention, the invention feature an element connecting the wire to the intravascular device that permits free rotation of the device relative to the wire over a defined angle. For example, the device can freely rotate relative to the wire approximately 10, 20, 30, 40, 50, 60, 70, 80, 90, 120, 145, 160, 180, 210, 240, 270, 300, 330, or slightly less than 360 degrees (e.g., the wire can rotate at least 10, 20, 30, 40, 50, 60, 70, 80, 90, 120, 145, 160, 180, 210, 240, 270, 300, 330, or slightly less than 360 degrees or at most 10, 20, 30, 40, 50, 60, 70, 80, 90, 120, 145, 160, 180, 210, 240, 270, 300, 330, or slightly less than 360 degrees).

In some embodiments, the connecting element and first and second wire are arranged as depicted in FIGS. 9D-9L. In these embodiments, the intravascular device can be attached to the “first wire” (i.e., the wire affixed to the connecting element) or the “second wire” (i.e., the wire connected to the element containing the second stop, e.g., the tether in FIG. 9D). In certain embodiments, the connecting element can be joined to the second wire by bending the first stop element (the letch in FIG. 9D) in to the space (or window) formed into the tether element (depicted in FIG. 9G). The bending of the first stop into the space formed into the tether prevents the first and second from disconnecting and prevents the free rotation of the second wire relative to the first wire beyond a distance (e.g., as described above). Other embodiments are described in the claims and summary of the invention.

Reference is made to FIG. 10. The shaft or plunger for use in connection with the device can, e.g., terminate in a loop (as depicted in FIG. 10) or, e.g., a screw. In embodiments where a loop is present, the loop can be generated by winding two wires together leaving a loop at the distal end (FIG. 10). The shaft or plunger can, e.g., include a radiopaque element. Furthermore, the shaft or plunger can feature a rectilinear (e.g., square) or curved (e.g., oval or circular) cross section. Differences in cross sectional shape can have advantageous properties with respect to controlling the positioning of the intra-vascular device within the aorta.

It will be appreciated by persons skilled in the art that embodiments of the invention are not limited by what has been particularly shown and described hereinabove. Rather the scope of at least one embodiment of the invention is defined by the claims below.

1. An intravascular device comprising a first wire, a second wire, and a connecting element; wherein:
a. said connecting element comprises a hollow cylindrical body defining an internal channel along a longitudinal axis;
b. said connecting element joins said first wire and said second wire;
c. said connecting element comprises a first stop element;
d. said second wire comprises a second stop element; and
e. said first stop element is configured to reversibly engage said second stop element such that said second wire is only able to freely rotate relative to said connecting element over a distance of between 10 and 360 degrees.

2. The intravascular device of claim 1, wherein said second wire is able to freely rotate relative to said connecting element over a distance of greater than 60 degrees.

3. The intravascular device of claim 1, wherein said second wire is able to freely rotate relative to said connecting element over a distance of greater than 120 degrees.

4. The intravascular device of claim 1, wherein said second wire is able to freely rotate relative to said connecting element over a distance of greater than 180 degrees.

5. The intravascular device of any of claims 1-4, wherein said second wire is not able to freely rotate relative to said connecting element over a distance greater than 300 degrees.

6. The intravascular device of claim 5, wherein said second wire is not able to freely rotate relative to said connecting element over a distance greater than 270 degrees.

7. The intravascular device of claim 6, wherein said second wire is not able to freely rotate relative to said connecting element over a distance greater than 210 degrees.

8. The intravascular device of any of claims 1-3, wherein said second wire is not able to freely rotate relative to said connecting element over a distance greater than 180 degrees.

9. The intravascular device of claim 8, wherein said second wire is not able to freely rotate relative to said connecting element over a distance greater than 150 degrees.

10. The intravascular device of any of claims 1-2, wherein said second wire is not able to freely rotate relative to said connecting element over a distance greater than 120 degrees.

11. The intravascular device of any of claims 1-2, wherein said second wire is not able to freely rotate relative to said connecting element over a distance greater than 90 degrees.

12. The intravascular device of claim 1, wherein said second wire is not able to freely rotate relative to said connecting element over a distance greater than 60 degrees.

13. The intravascular device of claim 1, wherein said second wire is not able to freely rotate relative to said connecting element over a distance greater than 45 degrees.

14. The intravascular device of claim 1, wherein said second wire is not able to freely rotate relative to said connecting element over a distance greater than 30 degrees.

15. The intravascular device of any of claims 1-14, wherein said hollow cylindrical body comprises a window having two edges substantially parallel to said longitudinal axis and said edges reversibly engage said second stop element thereby defining said first stop element.

16. The intravascular device of any of claims 1-14, wherein said first stop element is disposed on the interior of said hollow cylindrical body.

17. The intravascular device of any of claims 1-14, wherein said hollow cylindrical body has a window and said first stop element is disposed within said window.

18. The intravascular device of any of claims 1-17, wherein said second stop is a protrusion disposed on the surface of said second wire.

19. The intravascular device of any of claims 1-17, wherein said second stop element comprises a substantially cylindrical element that is disposed about said second wire or joined to an end of said second wire, said substantially cylindrical element comprising a window having two edges substantially parallel to said longitudinal axis that reversibly engages said first stop element.

20. The intravascular device of claim 17, wherein said second stop element comprises a substantially cylindrical element that is disposed about said second wire or joined to an end of said second wire, said substantially cylindrical element comprising a window comprising:
(i) two edges substantially parallel to said longitudinal axis that reversibly engage said first stop element; and
(ii) a third edge substantially orthogonal said longitudinal axis.

21. The intravascular device of claim 20, wherein said second wire and said connecting element are joined by bending said first stop such that it is disposed within said window in said substantially cylindrical element, thereby permitting reversible engagement with said second stop element.

22. The intravascular device of claim 21, wherein upon disposition within said window in said substantially cylindrical element, said second stop element reversibly engages said third edge, thereby preventing separation of said second wire and said connecting element.

23. A device for deflecting emboli, the device comprising:
a lateral structure to support an emboli filter, a length of said lateral structure being between 80 mm and 90 mm and a width of said lateral structure being from 20 mm to 35 mm, having the filter attached to and extending the length of said lateral structure;
a lower member extending downward from said lateral structure in a direction of an ascending aorta, wherein upon installation of said device, said lower member exerts lift on a middle area of said lateral structure; and
an upper member extending upwards from said lateral structure, wherein a support portion of said upper member being proximate to said lateral structure is angled towards the ascending aorta, and an anchor portion of said upper member being distal to said lateral structure is angled towards the descending aorta,
wherein upon said installation, said upper member limits said lift;
wherein said device comprises three or more radiopaque elements; and
wherein said three or more radiopaque elements are positioned asymmetrically.

24. The device of claim 23 wherein said three or more radiopaque elements are asymmetric in 2 or more axes.

25. The device of any one of claim 23 or 24, wherein one or more radiopaque elements is a clamp.

26. The device of any one of claim 23 or 24, wherein one or more radiopaque elements is a bead.

27. The device of any one of claim 23 or 24, wherein one or more radiopaque elements are incorporated into all or a portion of the wire forming the skeleton or filter mesh of said device.

28. The device of any of claims 23-27, wherein said one or more radiopaque elements are located exclusively at the following positions: a junction between the lateral structure and lower member, a junction between the lateral structure and upper member, at the top of said upper member, and, optionally, at the distal and proximate ends of said lateral structure.

29. The device of claim 28, wherein a radiopaque element is located at the distal end of said lateral structure.

30. The device of claim 28 or 29, wherein a radiopaque element is located at the proximal end of said lateral structure.
